(19) United States
(12) Patent Application Publication
(10) Pub. No.: US 2008/0171086 A1
(43) Pub. Date: Jul. 17, 2008

(54) Solid Dosage Forms of Valsartan and Amlo Dipine and Method of Making the Same

(76) Inventors: Yatindra Joshi, Princeton, NJ (US); Robert Frank Wagner, Hillsborough, NJ (US); Madhusudhan Pudipeddi, Mubai (IN); Gangadhar Sunkara, Parsippany, NJ (US); Ping Li, Basking Ridge, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/914,159

(22) PCT Filed: Aug. 15, 2006
(86) PCT No.: PCT/US06/31699

§ 371 Date: Nov. 12, 2007

Related U.S. Application Data
(60) Provisional application No. 60/709,083, filed on Aug. 17, 2005.

(51) Int. Cl. A61K 9/28; A61K 31/455; A61P 9/00; A61K 9/20

(52) U.S. Cl. 424/474; 514/356; 424/464

(57) ABSTRACT

Monolayer and bilayer solid dosage forms of a combination of valsartan and amlodipine are made.

### BACKGROUND OF THE INVENTION

1. Field of the Invention

The present invention is directed to solid dosage formulations containing a combination of valsartan and amlodipine, as well as to methods of making such solid dosage forms and a method of treating a subject with such solid dosage forms.

2. Related Background Art

The development of fixed-combination solid dosage formulations of certain active ingredients is challenging. As used herein, “fixed-combination” refers to a combination of two drugs or active ingredients presented in a single dosage unit such as a tablet or a capsule; further as used herein, “free-combination” refers to a combination of two drugs or active ingredients dosed simultaneously but as two dosage units. When formulating fixed-combination solid dosage formulations, the objective is to provide a patient-convenient combination dosage form of active ingredients that is bioequivalent to the corresponding free-combination of the same active ingredients. Development of fixed-combination dosage formulations that are bioequivalent to the free-combination is challenging due to the multiplicity of challenges arising from pharmacokinetic and pharmaceutical properties of the drugs sought to be combined.

For example, valsartan has an absolute oral bioavailability of only about 25% with a wide range of 10-35%. Valsartan also has pH dependent solubility whereby it ranges from very slightly soluble in an acidic environment to soluble in a neutral environment of the gastrointestinal tract. Further, development of a patient-convenient oral dosage form of valsartan is challenging due to its low bulk density. Amlodipine besylate is slightly soluble in water and has an absolute bioavailability of 64-90%. As a result of these complex biopharmaceutical properties, development of a fixed-combination dosage form of valsartan and amlodipine that is bioequivalent to a free-combination thereof is challenging.

Accordingly, a fixed-combination solid dosage formulation of valsartan and amlodipine that is bioequivalent to the corresponding free-combination would be desirable.

### SUMMARY OF THE INVENTION

In a first aspect, the present invention is directed to a solid dosage form comprising a combination of valsartan and amlodipine, and pharmaceutically acceptable additives suitable for the preparation of solid dosage forms of valsartan. In preferred embodiments of this invention, amlodipine free base is provided in the form of amlodipine besylate, and the pharmaceutically acceptable additives are selected from diluents, disintegrants, glidants, lubricants, colorants and combinations thereof.

In certain preferred embodiments of this invention, the solid dosage form is a monolayer tablet. The amount of valsartan employed in such monolayer tablets preferably ranges from about 40 mg to about 640 mg, and more preferably is 80 mg or 160 mg. The amount of amlodipine employed in such monolayer tablets preferably ranges from about 1.25 mg to about 20 mg, and more preferably is 2.5 mg, 5 mg or 10 mg.

In other preferred embodiments of this invention, the solid dosage form is a bilayer tablet having the valsartan in one layer and the amlodipine in another layer. The amount of valsartan employed in such bilayer tablets preferably ranges from about 40 mg to about 640 mg, and more preferably is 320 mg. The amount of amlodipine employed in such bilayer tablets preferably ranges from about 1.25 mg to about 20 mg, and more preferably is 5 mg or 10 mg.

In a second aspect, the present invention is directed to a method of making a solid dosage form of valsartan comprising the steps of (a) blending valsartan, amlodipine and pharmaceutically acceptable additives to form a blended material; (b) sieving the blended material to form a sieved material; (c) blending the sieved material to form a blended/sieved material; (d) compacting the blended/sieved material to form a compacted material; (e) milling the compacted material to form a milled material; (f) blending the milled material to form blended/milled material; and (g) compressing the blended/milled material to form a monolayer solid dosage form. A preferred embodiment of this invention also includes an optional step, step (h) film coating the monolayer solid dosage form.

In a third aspect, this invention is directed to solid dosage forms of valsartan made according to the method of the second aspect.

In a fourth aspect, the present invention is directed to a method of making a solid dosage form of valsartan comprising the steps of (a) granulating valsartan and pharmaceutically acceptable additives to form a valsartan granulation; (b) blending amlodipine and pharmaceutically acceptable additives to form an amlodipine blend; and (c) compressing the valsartan granulation and the amlodipine blend together to form a bilayer solid dosage form. In a preferred embodiment of the invention, step (a) comprises the steps of (a1) blending valsartan and pharmaceutically acceptable additives to form a blended material; (a2) sieving the blended material to form a sieved material; (a3) blending the sieved material to form a blended/sieved material; (a4) compacting the blended/sieved material to form a compacted material; (a5) milling the compacted material to form a milled material; and (a6) blending the milled material to form the valsartan granulation. In another preferred embodiment, step (b) comprises a granulation process with the steps of (b1) blending amlodipine and pharmaceutically acceptable additives to form a blended material; (b2) sieving the blended material to form a sieved material; (b3) blending the sieved material to form a blended/sieved material; (b4) compacting the blended/sieved material to form a compacted material; (b5) milling the compacted material to form a milled material; and (b6) blending the milled material to form an amlodipine granulation. Another preferred embodiment of this invention also includes an optional step, step (d) film coating the bilayer solid dosage form.

In a fifth aspect, this invention is directed to solid dosage forms of valsartan made according to the method of the fourth aspect.

Yet another aspect of the invention is directed to a method of treating hypertension, congestive heart failure, angina, myocardial infarction, arteriosclerosis, diabetic nephropathy, diabetic cardiac myopathy, renal insufficiency, peripheral vascular disease, stroke, left ventricular hypertrophy, cognitive dysfunction, headache, or chronic heart failure comprising administering a solid dosage form of valsartan and amlodipine to a subject in need of such treatment. In a preferred embodiment, the solid dosage form is orally administered to the subject.

### DETAILED DESCRIPTION

The present invention relates to solid dosage forms of valsartan which contain a combination of valsartan and amlodipine.

The first embodiment of the invention is directed to a solid dosage form of valsartan comprising a combination of valsartan and amlodipine, and pharmaceutically acceptable additives suitable for the preparation of solid dosage forms of valsartan. The solid dosage forms of the present invention can take the form of monolayer tablets (having both the valsartan and the amlodipine in one layer) or bilayer tablets (having the valsartan in one layer and the amlodipine in another layer).

Valsartan ((S)-N-valeryl-N-{[2′-(1H-tetrazole-5-yl)-biphenyl-4-yl]-methyl}-valine) suitable for use in the present invention can be purchased from commercial sources or can be prepared according to known methods. For example, the preparation of valsartan is described in U.S. Pat. No. 5,399,578, the entire disclosure of which is incorporated by reference herein. Valsartan may be used for purposes of this invention in its free form as well as in any suitable salt form.

Valsartan is employed in an amount typically ranging from about 40 mg to about 640 mg, preferably from about 40 mg to about 320 mg, more preferably from about 80 mg to about 320 mg, and most preferably is about 80 mg or about 160 mg in a monolayer tablet and about 320 mg in a bilayer tablet. The amount of valsartan noted above refers to the amount of free valsartan present in a given solid dosage form.

While both monolayer and bilayer tablets can be formed with any amount of valsartan within the above-noted range, it is important to consider the overall objective of bioequivalence to the free-combination of valsartan and amlodipine. Accordingly, monolayer tablets preferably contain a dose of up to 160 mg of valsartan; higher doses therein do not yield complete bioequivalence when compared with a corresponding free-combination. Hence, valsartan doses higher than 160 mg are better suited for bilayer solid dosage forms of the present invention. In fact, bilayer tablets can accommodate the full range of valsartan dosage above. It should be noted, however, that changes in the composition, i.e., a change in the type of disintegrant, may modify the dissolution properties of valsartan and achieve bioequivalence even at higher doses in monolayer tablets.

Amlodipine (3-ethyl-5-methyl-2(2-aminoethoxymethyl)-4-(2-chlrorophenyl)-1,4-dihydro-6-methyl-3,5-pyridinedicarboxylate benzenesulphonate) suitable for use in the present invention can be purchased from commercial sources or can be prepared according to known methods. Amlodipine may be used for purposes of this invention in its free form as well as in any suitable salt form; in a preferred embodiment of this invention, amlodipine free base is supplied to the solid dosage forms through the use of amlodipine besylate.

Amlodipine is employed in an amount ranging from 1.25 mg to about 20 mg, preferably from about 1.875 mg to about 15 mg, more preferably from about 2.5 mg to about 10 mg, and most preferably is about 2.5 mg or about 5 mg in a monolayer tablet and about 5 mg or about 10 mg in a bilayer tablet. The amount of amlodipine noted above refers to the amount of free amlodipine present in a given solid dosage form.

Pharmaceutically acceptable additives suitable for use in the present invention include, without limitation, diluents or fillers, disintegrants, glidants, lubricants, binders, colorants and combinations thereof. Preferred pharmaceutically acceptable additives include diluents and disintegrants. The amount of each additive in a solid dosage formulation may vary within ranges conventional in the art.

Suitable diluents include, without limitation, microcrystalline cellulose (e.g., cellulose MK GR), mannitol, sucrose or other sugars or sugar derivatives, low-substituted hydroxypropyl cellulose, and combinations thereof. When present, a diluent may be employed in an amount ranging from about 15% to about 70%, preferably from about 32% to about 55% by weight of the solid dosage form (prior to any optional film coating). For monolayer tablets, a diluent is preferably employed in an amount ranging from about 15% to about 50%, more preferably in an amount of about 33% by weight of the solid dosage form. For bilayer tablets, a diluent is preferably employed in an amount ranging from about 40% to about 70%, more preferably in an amount of about 55% by weight of the solid dosage form.

Suitable disintegrants include, without limitation, crospovidone, sodium starch glycolate, L-hydroxy propyl cellulose, and combinations thereof. When present, a disintegrant may be employed in an amount ranging from about 2% to about 40%, preferably from about 7% to about 13% by weight of the solid dosage form (prior to any optional film coating). For monolayer tablets, a disintegrant is preferably employed in an amount ranging from about 2% to about 40%, more preferably in an amount of about 13% by weight of the solid dosage form. For bilayer tablets, a disintegrant is preferably employed in an amount ranging from about 2% to about 40%, more preferably in an amount of about 7% by weight of the solid dosage form.

Suitable glidants include, without limitation, colloidal silicon dioxide (e.g., Aerosil 200), magnesium trisilicate, powdered cellulose, starch, talc and combinations thereof. When present, a glidant may be employed in an amount ranging from about 0.1% to about 10%, preferably from about 0.6% to about 1% by weight of the solid dosage form (prior to any optional film coating). For monolayer tablets, a glidant is preferably employed in an amount ranging from about 0.1% to about 10%, more preferably in an amount of about 1% by weight of the solid dosage form. For bilayer tablets, a glidant is employed in an amount ranging from about 0.1% to about 10%, more preferably in an amount of about 0.7% by weight of the solid dosage form.

Suitable lubricants include, without limitation, magnesium stearate, aluminum or calcium silicate, stearic acid, cutina, PEG 4000-8000, talc and combinations thereof. When present, a lubricant may be employed in an amount ranging from about 0.1% to about 5%, preferably from about 2% to about 3% by weight of the solid dosage form (prior to any optional film coating). For monolayer tablets, a lubricant is preferably employed in an amount ranging from about 0.1% to about 5%, more preferably in an amount of about 3% by weight of the solid dosage form. For bilayer tablets, a lubricant is preferably employed in an amount ranging from about 0.1% to about 5%, more preferably in an amount of about 2% by weight of the solid dosage form.

Suitable binders include, without limitation, polyvinylpyrrolidone, hydroxypropylmethyl cellulose, hydroxypropyl cellulose, pregelatinized starch and combinations thereof. When present, a binder may be employed in an amount ranging from about 2% to about 40%, preferably from about 7% to about 13% by weight of the solid dosage form (prior to any optional film coating). For monolayer tablets, a binder is preferably employed in an amount ranging from about 2% to about 40%, more preferably in an amount of about 13% by weight of the solid dosage form. For bilayer tablets, a binder is preferably employed in an amount ranging from about 2% to about 40%, more preferably in an amount of about 7% by weight of the solid dosage form.

Suitable colorants include, without limitation, iron oxides such as yellow, white, red, and black iron oxide, and combinations thereof. When present, a colorant may be employed in an amount ranging from about 0.01% to about 0.1% by weight of the solid dosage form (prior to any optional film coating). In a preferred embodiment, monolayer tablets contain no colorant.

The solid dosage forms of the first embodiment of the invention are monolayer or bilayer tablet dosage forms of suitable hardness (e.g., an average hardness ranging from about 30 N to about 180 N for monolayer forms and an average hardness ranging from about 250 N to about 300 N for bilayer forms). Such an average hardness is determined prior to the application of any film coating on the solid dosage forms. In that regard, a preferred embodiment of this invention is directed to solid dosage forms which are film-coated. Suitable film coatings are known and commercially available or can be made according to known methods. Typically the film coating material is a polymeric film coating material comprising materials such as hydroxypropylmethyl cellulose, polyethylene glycol, talc and colorant. Typically, a film coating material is applied in such an amount as to provide a film coating that ranges from about 1% to about 6% by weight of the film-coated tablet.

The second embodiment of the present invention is directed to a method of making a solid dosage form of valsartan comprising the steps of (a) blending valsartan, amlodipine and pharmaceutically acceptable additives to form a blended material; (b) sieving the blended material to form a sieved material; (c) blending the sieved material to form a blended/sieved material; (d) compacting the blended/sieved material to form a compacted material; (e) milling the compacted material to form a milled material; (f) blending the milled material to form blended/milled material; and (g) compressing the blended/milled material to form a monolayer solid dosage form. The details regarding the valsartan, amlodipine, and pharmaceutically acceptable additives, i.e., source, amount, etc., are as set forth above with regard to the first embodiment of the invention.

In the first step of the method of the second embodiment, valsartan, amlodipine and pharmaceutically acceptable additives are blended to form a blended material. Blending can be accomplished using any suitable means such as a diffusion blender or diffusion mixer. In the second step, the blended material is sieved to form a sieved material. Sieving can be accomplished using any suitable means. In the third step of the method of the second embodiment, the sieved material is blended to form a blended/sieved material. Again blending can be accomplished using any suitable means.

In the fourth step, the blended/sieved material is compacted to form a compacted material. Compacting can be accomplished using any suitable means. Typically compacting is accomplished using a roller compactor with a compaction force ranging from about 20 kN to about 60 kN, preferably about 30 kN to about 40 KN. Compaction may also be carried out by slugging the blended powders into large tablets that are then size-reduced.

In the fifth step of the method of the second embodiment, the compacted material is milled to form a milled material. Milling can be accomplished using any suitable means. In the sixth step, the milled material is blended to form blended/milled material. Here again blending can be accomplished using any suitable means. In the final step of the method of the second embodiment, the blended/milled material is compressed to form a monolayer solid dosage form. Compression can be accomplished using any suitable means. Typically compression is accomplished using a rotary tablet press. Compression force for such a rotary tablet press typically ranges from about 2 kN to about 30 kN.

Optionally, the method of the second embodiment comprises the step of (h) film coating the monolayer solid dosage form. The details regarding the film coating material, i.e., components, amounts, etc., are as described above with regard to the first embodiment of the invention. Film coating can be accomplished using any suitable means.

A third embodiment of the present invention is directed to a monolayer solid dosage form of valsartan made according to the method of the second embodiment.

The fourth embodiment of the present invention is directed to a method of making a solid dosage form of valsartan comprising the steps of (a) granulating valsartan and pharmaceutically acceptable additives to form a valsartan granulation; (b) blending amlodipine and pharmaceutically acceptable additives to form an amlodipine blend; and (c) compressing the valsartan granulation and the amlodipine blend together to form a bilayer solid dosage form. The details regarding the valsartan, amlodipine, and pharmaceutically acceptable additives, i.e., source, amount, etc., are as set forth above with regard to the first embodiment of the invention.

In the first step of the method of the fourth embodiment, valsartan is granulated with pharmaceutically acceptable additives to form a valsartan granulation. Valsartan granulation can be accomplished by any suitable means. In a preferred embodiment of this invention, valsartan granulation is accomplished by (a1) blending valsartan and pharmaceutically acceptable additives to form a blended material; (a2) sieving the blended material to form a sieved material; (a3) blending the sieved material to form a blended/sieved material; (a4) compacting the blended/sieved material to form a compacted material; (a5) milling the compacted material to form a milled material; and (a6) blending the milled material to form the valsartan granulation.

The blending of step (a1) can be accomplished using any suitable means. Typically the valsartan and pharmaceutically acceptable additives are dispatched to a suitable vessel such as a diffusion blender or diffusion mixer. The sieving of step (a2) can be accomplished using any suitable means. The blending of step (a3) can be accomplished using any suitable means. The compacting of step (a4) can be accomplished using any suitable means. Typically compacting is accomplished using a roller compactor with a compaction force ranging from about 20 kN to about 60 kN, preferably about 35 kN. Compaction may also be carried out by slugging the blended powders into large tablets that are then size-reduced. The milling of step (a5) can be accomplished using any suitable means. Typically the compacted material is milled through a screening mill. The blending of step (a6) can be accomplished using any suitable means. Preferably the milled material is blended, often with a pharmaceutically acceptable additive such as a lubricant, in a diffusion blender.

In the second step of the method of the fourth embodiment, amlodipine is blended with pharmaceutically acceptable additives to form an amlodipine blend. Amlodipine blending can be accomplished by any suitable means. In a preferred embodiment, blending step (b) comprises the process of granulating amlodipine. Amlodipine granulation can be accomplished by any suitable means including wet granulation or dry granulation. In a more preferred embodiment of this invention, amlodipine granulation is accomplished by (b1) blending amlodipine and pharmaceutically acceptable additives to form a blended material; (b2) sieving the blended material to form a sieved material; (b3) blending the sieved material to form a blended/sieved material; (b4) compacting the blended/sieved material to form a compacted material; (b5) milling the compacted material to form a milled material; and (b6) blending the milled material to form an amlodipine granulation.

The blending of step (b1) can be accomplished using any suitable means. The sieving of step (b2) can be accomplished using any suitable means. The blending of step (b3) can be accomplished using any suitable means. The compacting of step (b4) can be accomplished using any suitable means. Typically compacting is accomplished using a roller compactor with a compaction force ranging from about 20 kN to about 50 kN, preferably about 30 kN to about 40 kN. The milling of step (b5) can be accomplished using any suitable means. Typically the compacted material is milled through a screening mill. The blending of step (b6) can be accomplished using any suitable means.

In the final step of the method of the fourth embodiment, the valsartan granulation and the amlodipine blend are compressed together to form a bilayer solid dosage form. Compression can be accomplished using any suitable means. Typically compression is accomplished using a bilayer rotary tablet press. Typical compression force ranges from about 5 kN to about 35 kN.

Optionally, the method of the fourth embodiment comprises the step of (d) film coating the bilayer solid dosage form. The details regarding the film coating material, i.e., components, amounts, etc., are as described above with regard to the first embodiment of the invention. Film coating can be accomplished using any suitable means.

A fifth embodiment of the present invention is directed to a bilayer solid dosage form of valsartan made according to the method of the fourth embodiment.

Yet another embodiment of the invention is directed to a method of treating hypertension, congestive heart failure, angina, myocardial infarction, arteriosclerosis, diabetic nephropathy, diabetic cardiac myopathy, renal insufficiency, peripheral vascular disease, stroke, left ventricular hypertrophy, cognitive dysfunction, headache, or chronic heart failure. The method comprises administering a solid dosage form of valsartan as defined by the first, third or fifth embodiments of this invention to a subject in need of such treatment. In a preferred embodiment, the solid dosage form is orally administered to the subject.

Specific embodiments of the invention will now be demonstrated by reference to the following examples. It should be understood that these examples are disclosed solely by way of illustrating the invention and should not be taken in any way to limit the scope of the present invention.

### EXAMPLE 1 80/2.5 MG TABLET

A monolayer solid dosage form of valsartan was made using the ingredients set forth in Table 1 below.

TABLE 1

| | Ingredient | (mg) | % |
|---|---|---|---|
| A | valsartan | 80.00 | 48.78 |
| B | amlodipine besylate | 3.47* | 2.11** |
| C | microcrystalline cellulose | 54.53 | 33.25 |
| D | crospovidone | 20.00 | 12.20 |
| E | colloidal silicon dioxide | 1.50 | 0.91 |
| F | magnesium stearate (I) | 3.00 | 1.83 |
| G | magnesium stearate (II) | 1.50 | 0.91 |
| | total | 164.00 | |

*corresponds to 2.5 mg amlodipine free base
**corresponds to 1.52% amlodipine free base

Ingredients A-F are placed into a diffusion blender and blended. Then, the blended material is sieved. Next, the sieved material is blended again in a diffusion blender. The blended/sieved material is then compacted using a roller compactor. The compacted material is milled through a screen and then blended with ingredient G in a diffusion blender. (This second blending step achieves the desired level of lubricant for the granulation and, in certain cases, combines sub-divided batches of ingredients A-F.) Next, the blended/milled material is compressed into monolayer solid dosage forms using a rotary tablet press, and the monolayer solid dosage forms are optionally film coated.

### EXAMPLE 2 80/5 MG TABLET

A monolayer solid dosage form of valsartan was made using the ingredients set forth in Table 2 below.

TABLE 2

| | Ingredient | (mg) | % |
|---|---|---|---|
| A | valsartan | 80.00 | 47.90 |
| B | amlodipine besylate | 6.94* | 4.15** |
| C | microcrystalline cellulose | 54.06 | 32.37 |
| D | crospovidone | 20.00 | 11.98 |
| E | colloidal silicon dioxide | 1.50 | 0.90 |
| F | magnesium stearate (I) | 3.00 | 1.80 |
| G | magnesium stearate (II) | 1.50 | 0.90 |
| | total | 167.00 | |

*corresponds to 5 mg amlodipine free base
**corresponds to 2.99% amlodipine free base

Ingredients A-F are placed into a diffusion blender and blended. Then, the blended material is sieved. Next, the sieved material is blended again in a diffusion blender. The blended/sieved material is then compacted using a roller compactor. The compacted material is milled through a screen and then blended with ingredient G in a diffusion blender. (This second blending step achieves the desired level of lubricant for the granulation and, in certain cases, combines sub-divided batches of ingredients A-F.) Next, the blended/milled material is compressed into monolayer solid dosage forms using a rotary tablet press, and the monolayer solid dosage forms are optionally film coated.

### EXAMPLE 3 160/5 MG TABLET

A monolayer solid dosage form of valsartan was made using the ingredients set forth in Table 3 below.

TABLE 3

| | Ingredient | (mg) | % |
|---|---|---|---|
| A | valsartan | 160.00 | 48.78 |
| B | amlodipine besylate | 6.94* | 2.11** |
| C | microcrystalline cellulose | 109.06 | 33.25 |
| D | crospovidone | 40.00 | 12.20 |
| E | colloidal silicon dioxide | 3.00 | 0.91 |
| F | magnesium stearate (I) | 6.00 | 1.83 |
| G | magnesium stearate (II) | 3.00 | 0.91 |
| | total | 328.00 | |

*corresponds to 5 mg amlodipine free base
**corresponds to 1.52% amlodipine free base

Ingredients A-F are placed into a diffusion blender and blended. Then, the blended material is sieved. Next, the sieved material is blended again in a diffusion blender. The blended/sieved material is then compacted using a roller compactor. The compacted material is milled through a screen and then blended with ingredient G in a diffusion blender. (This second blending step achieves the desired level of lubricant for the granulation and, in certain cases, combines sub-divided batches of ingredients A-F.) Next, the blended/milled material is compressed into monolayer solid dosage forms using a rotary tablet press, and the monolayer solid dosage forms are optionally film coated.

### EXAMPLE 4 160/10 MG TABLET

A monolayer solid dosage form of valsartan was made using the ingredients set forth in Table 4 below.

TABLE 4

| | ingredient | (mg) | % |
|---|---|---|---|
| A | valsartan | 160.00 | 47.90 |
| B | amlodipine besylate | 13.87* | 4.15** |
| C | microcrystalline cellulose | 108.13 | 32.37 |
| D | crospovidone | 40.00 | 11.98 |
| E | colloidal silicon dioxide | 3.00 | 0.90 |
| F | magnesium stearate (I) | 6.00 | 1.80 |
| G | magnesium stearate (II) | 3.00 | 0.90 |
| | total | 334.00 | |

*corresponds to 10 mg amlodipine free base
**corresponds to 2.99% amlodipine free base

Ingredients A-F are placed into a diffusion blender and blended. Then, the blended material is sieved through a screen. Next, the sieved material is blended again in a diffusion blender. The blended/sieved material is then compacted using a roller compactor. The compacted material is milled through a screen and then blended with ingredient G in a diffusion blender. (This second blending step achieves the desired level of lubricant for the granulation and, in certain cases, combines sub-divided batches of ingredients A-F.) Next, the blended/milled material is compressed into monolayer solid dosage forms using a rotary tablet press, and the monolayer solid dosage forms are optionally film coated.

### EXAMPLE 5 320/5 MG TABLET

A monolayer solid dosage form of valsartan was made using the ingredients set forth in Table 5 below.

TABLE 5

| | ingredient | (mg) | % |
|---|---|---|---|
| A | valsartan | 320.00 | 49.46 |
| B | amlodipine besylate | 6.94* | 1.07 |
| C | microcrystalline cellulose | 216.07 | 33.40 |
| D | crospovidone | 80.00 | 12.36 |
| E | colloidal silicon dioxide | 6.00 | 0.93 |
| F | magnesium stearate (I) | 12.00 | 1.85 |
| G | magnesium stearate (II) | 6.00 | 0.93 |
| | total | 647.00 | |

*corresponds to 5 mg amlodipine free base

Ingredients A-F are placed into a diffusion blender and blended. Then, the blended material is sieved through screen. Next, the sieved material is blended again in a diffusion blender. The blended/sieved material is then compacted using a roller compactor. The compacted material is milled through a screen and then blended with ingredient G in a diffusion blender. (This second blending step achieves the desired level of lubricant for the granulation and, in certain cases, combines sub-divided batches of ingredients A-F.) Next, the blended/milled material is compressed into monolayer solid dosage forms using a rotary tablet press, and the monolayer solid dosage forms are optionally film coated.

### EXAMPLE 6 320/5 MG TABLET

A bilayer solid dosage form of valsartan was made using the ingredients set forth in Table 6 below.

TABLE 6

| | | |
|---|---|---|
| Ingredient | (mg) | % |

valsartan layer

| | | | |
|---|---|---|---|
| A | valsartan | 320.00 | 34.78 |
| B | microcrystalline cellulose | 216.00 | 23.48 |
| C | crospovidone | 60.00 | 6.52 |
| D | colloidal silicon dioxide | 6.00 | 0.65 |
| E | magnesium stearate (I) | 12.00 | 1.30 |
| F | magnesium stearate (II) | 6.00 | 0.65 |
| | subtotal | 620.00 | |

amlodipine layer

| | | | |
|---|---|---|---|
| G | amlodipine besylate | 6.94* | 0.75 |
| H | microcrystalline cellulose | 285.96 | 31.08 |
| I | sodium starch glycolate | 6.00 | 0.65 |
| J | colorant | 0.20 | 0.02 |
| K | magnesium stearate (III) | 0.30 | 0.03 |
| L | magnesium stearate (IV) | 0.60 | 0.07 |
| | subtotal | 300.00 | |
| | total | 920.00 | |

*corresponds to 5 mg amlodipine free base

First, the valsartan is granulated by combining ingredients A-E in a diffusion blender. Then, the blended material is sieved through a screen. Next, the sieved material is blended again in a diffusion blender. The blended/sieved material is then compacted using a roller compactor. The compacted material is milled through a screen and then blended with ingredient F in a diffusion blender. (This second blending step achieves the desired level of lubricant for the granulation and, in certain cases, combines sub-divided batches of ingredients A-E.)

Second, the amlodipine besylate is granulated by combining ingredients G-K in a diffusion blender. Then, the blended material is sieved through a screen. Next, the sieved material is blended again in a diffusion blender. The blended/sieved material is then compacted using a roller compactor. The compacted material is milled through a screen and then blended with ingredient L in a diffusion blender. (This second blending step achieves the desired level of lubricant for the granulation and, in certain cases, combines sub-divided batches of ingredients G-K.)

Finally, the valsartan granulation and the amlodipine granulation are compressed into bilayer solid dosage forms using a bilayer rotary tablet press, and the bilayer solid dosage forms are optionally film coated.

### EXAMPLE 7 320/10 MG TABLET

A bilayer solid dosage form of valsartan was made using the ingredients set forth in Table 7 below.

TABLE 7

| | | |
|---|---|---|
| Ingredient | (mg) | % |

valsartan layer

| | | | |
|---|---|---|---|
| A | valsartan | 320.00 | 34.78 |
| B | microcrystalline cellulose | 216.00 | 23.48 |
| C | crospovidone | 60.00 | 6.52 |
| D | colloidal silicon dioxide | 6.00 | 0.65 |
| E | magnesium stearate (I) | 12.00 | 1.30 |
| F | magnesium stearate (II) | 6.00 | 0.65 |
| | Subtotal | 620.00 | |

amlodipine layer

| | | | |
|---|---|---|---|
| G | amlodipine besylate | 13.87* | 1.51 |
| H | microcrystalline cellulose | 279.03 | 30.33 |
| I | sodium starch glycolate | 6.00 | 0.65 |
| J | Colorant | 0.20 | 0.02 |
| K | magnesium stearate (III) | 0.30 | 0.03 |
| L | magnesium stearate (IV) | 0.60 | 0.07 |
| | Subtotal | 300.00 | |
| | Total | 920.00 | |

*corresponds to 10 mg amlodipine free base

First, the valsartan is granulated by combining ingredients A-E in a diffusion blender. Then, the blended material is sieved through a screen. Next, the sieved material is blended again in a diffusion blender. The blended/sieved material is then compacted using a roller compactor. The compacted material is milled through a screen and then blended with ingredient F in a diffusion blender. (This second blending step achieves the desired level of lubricant for the granulation and, in certain cases, combines sub-divided batches of ingredients A-E.)

Second, the amlodipine besylate is granulated by combining ingredients G-K in a diffusion blender. Then, the blended material is sieved through a screen. Next, the sieved material is blended again in a diffusion blender. The blended/sieved material is then compacted using a roller compactor. The compacted material is milled through a screen and then with ingredient L in a diffusion blender. (This second blending step achieves the desired level of lubricant for the granulation and, in certain cases, combines sub-divided batches of ingredients G-K.)

Finally, the valsartan granulation and the amlodipine granulation are compressed into bilayer solid dosage forms using a bilayer rotary tablet press, and the bilayer solid dosage forms are optionally film coated.

### BIOEQUIVALENCE TESTING

The bioavailability of the fixed-combination dosage forms of the present invention was compared with that of the corresponding free-combinations. The term “bioavailability”, as used herein, is defined as a measure of the rate and amount of drug which reaches the systemic circulation unchanged following the administration of the dosage form. The test (fixed-combination) and the reference (free-combination) dosage forms were administered orally to the subjects, and plasma samples were collected over a 48-hour time period. The plasma samples were analyzed for concentration of valsartan and amlodipine. Statistical comparison was performed on the maximum plasma concentration (Cmax) achieved with the test and reference and on the area under the plasma concentration vs. time curve (AUC). For the test and reference product to be bioequivalent, 90% confidence intervals for AUC and Cmax ratios should fall within 0.8-1.25.

Obtaining bioequivalence between test and reference products is challenging, particularly for combinations of drugs, and the result cannot be predicted apriori. The challenge of bioequivalence is more pronounced if one or more drugs has solubility limitations and variable absorption (e.g., valsartan). Comparison of dissolution (release of the drugs from the dosage forms into a solvent medium such as an aqueous buffer) is often used to guide dosage development to obtain bioequivalence. However, drug dissolution (in vitro dissolution) may not fully correlate with in vivo (in animal or human body) drug absorption. This invention is directed to solid dosage formulations containing valsartan and amlodipine in a fixed-combination form that is bioequivalent to the free-combination.

A fixed-combination solid dosage form (monolayer tablet) of valsartan and amlodipine besylate (160/10 mg) made in accordance with Example 4 was compared with a free-combination of 160 mg valsartan and 10 mg amlodipine besylate tablets in an open-label, randomized, single dose, three period, crossover study in twenty-seven healthy human volunteers. It was found that the in vitro dissolution of valsartan from the fixed-combination monolayer tablets, when tested using a USP II apparatus in a test medium such as pH 4.5 phosphate solution or pH 6.8 phosphate solution, was similar to the dissolution of valsartan in the free-combination. The difference between the percent of valsartan dissolved from the fixed- and free-combination dosage formulations was no more than 10% at 10, 20, or 30 minutes, by which time the dissolution was nearly complete. However, the dissolution of amlodipine from the fixed-combination tablet was different from that of amlodipine in the free-combination in both pH 4.5 and 6.8 media. In pH 6.8 phosphate solution, amlodipine in the fixed-combination dosage form dissolved faster by about 30% fraction dissolved at 30 minutes, for example. In pH 4.5 phosphate solution, the dissolution of amlodipine from the fixed-combination was slower than the dissolution of amlodipine as a free-combination by about 35% fraction dissolved at 30 minutes, for example. Surprisingly, however, when the bioavailability of the fixed-combination tablets of valsartan and amlodipine besylate were compared with the free-combination, the 90% confidence interval for AUC and Cmax ratios were within the interval of 0.80-1.25 for amlodipine. This result indicated that amlodipine in the fixed-combination dosage form was bioequivalent to that in the free-combination. For valsartan, the 90% confidence interval for AUC fell within 0.80-1.25. The 90% confidence interval for Cmax was 0.77-1.21, only slightly outside of 0.80 on the lower limit. The ratio of the means of Cmax from the fixed- and free-combinations was very close (0.97). This result indicated that valsartan from the fixed-combination was nearly bioequivalent to that in the free-combination, and, by increasing the number of subjects, the statistical 90% interval range of 0.8-1.25 may be achieved for Cmax also.

In addition, a fixed-combination solid dosage form (monolayer tablet) of valsartan and amlodipine besylate (320/5 mg) made in accordance with Example 5 was compared with a free-combination of 320 mg valsartan and 5 mg amlodipine besylate tablets in an open-label, randomized, single dose, three period, crossover study in healthy human volunteers. It was found that the in vitro dissolution of valsartan from the fixed-combination monolayer tablets, when tested using a USP II apparatus in a test medium such as pH 4.5 phosphate solution or pH 6.8 phosphate solution, was similar to the dissolution of valsartan in the free-combination. The difference between the percent of valsartan dissolved from the fixed- and free-combination dosage formulations was no more than 10% at 10, 20, or 30 minutes, by which time the dissolution was nearly complete. However, the dissolution of amlodipine from the fixed-combination tablet was different from that of amlodipine in the free-combination in both pH 4.5 and 6.8 media. In pH 6.8 phosphate solution, amlodipine in the fixed combination dosage form dissolved faster by about 15% fraction dissolved at 30 minutes, for example. In pH 4.5 phosphate solution, the dissolution of amlodipine from the fixed -combination was slower than the dissolution of amlodipine as a free-combination by about 45% fraction dissolved at 30 minutes, for example. Very surprisingly, when the bioavailability of the fixed-combination tablets of valsartan and amlodipine besylate were compared with the free-combination, the 90% confidence interval for AUC and Cmax geometric mean ratios were within the interval of 0.80-1.25 for amlodipine. This result indicated that amlodipine in the fixed-combination dosage form was bioequivalent to that in the free-combination. For valsartan, the 90% confidence interval for AUC was 0.77-0.99. The 90% confidence interval for Cmax was 0.63-0.98. This result indicated that valsartan from the fixed-combination was not bioequivalent to that in the free-combination. However, this result led to another aspect of the inspection, namely the development of a bilayer fixed-combination dosage formulation of valsartan as shown in Examples 6 and 7.

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims. All patent applications, patents, and other publications cited herein are incorporated by reference in their entirety.

What is claimed is:

1. A solid dosage form of valsartan comprising:
valsartan;
amlodipine; and
pharmaceutically acceptable additives suitable for the preparation of solid dosage forms of valsartan.

2. The solid dosage form of claim 1, wherein the amlodipine is provided in the form of amlodipine besylate.

3. The solid dosage form of claim 1, wherein the solid dosage form takes the form of a monolayer tablet.

4. The solid dosage form of claim 1, wherein the valsartan is employed in an amount ranging from about 40 mg to about 640 mg.

5. The solid dosage form of claim 4, wherein the valsartan is employed in an amount selected from 80 mg and 160 mg.

6. The solid dosage form of claim 3, wherein the amlodipine is employed in an amount ranging from about 1.25 mg to about 20 mg.

7. The solid dosage form of claim 6, wherein the amlodipine is employed in an amount selected from 2.5 mg, 5 mg and 10 mg.

8. The solid dosage form of claim 1, wherein the solid dosage form takes the form of a bilayer tablet having the valsartan in a first layer and the amlodipine in a second layer.

9. The solid dosage form of claim 8, wherein the valsartan is employed in an amount ranging from about 40 mg to about 640 mg.

10. The solid dosage form of claim 9, wherein the valsartan is employed in an amount of 320 mg.

11. The solid dosage form of claim 8, wherein the amlodipine is employed in an amount ranging from about 1.25 mg to about 20 mg.

12. The solid dosage form of claim 11, wherein the amlodipine is employed in an amount selected from 5 mg and 10 mg.

13. The solid dosage form of claim 1, wherein the pharmaceutically acceptable additives are selected from the group consisting of diluents, disintegrants, glidants, lubricants, colorants and combinations thereof.

14. A method of making a solid dosage form of valsartan comprising the steps of:
(a) blending valsartan, amlodipine and pharmaceutically acceptable additives to form a blended material;
(b) sieving the blended material to form a sieved material;
(c) blending the sieved material to form a blended/sieved material;
(d) compacting the blended/sieved material to form a compacted material;
(e) milling the compacted material to form a milled material;
(f) blending the milled material to form blended/milled material; and
(g) compressing the blended/milled material to form a monolayer solid dosage form.

15. The method of claim 14 further comprising the step of:
(h) film coating the monolayer solid dosage form.

16. A method of making a solid dosage form of valsartan comprising the steps of:
(a) granulating valsartan and pharmaceutically acceptable additives to form a valsartan granulation;
(b) blending amlodipine and pharmaceutically acceptable additives to form an amlodipine blend; and
(c) compressing the valsartan granulation and the amlodipine blend together to form a bilayer solid dosage form.

17. The method of claim 16, wherein step (a) comprises the steps of:
(a1) blending valsartan and pharmaceutically acceptable additives to form a blended material;
(a2) sieving the blended material to form a sieved material;
(a3) blending the sieved material to form a blended/sieved material;
(a4) compacting the blended/sieved material to form a compacted material;
(a5) milling the compacted material to form a milled material; and
(a6) blending the milled material to form the valsartan granulation.

18. The method of claim 16, wherein step (b) comprises a granulation process comprising the steps of:
(b1) blending amlodipine and pharmaceutically acceptable additives to form a blended material;
(b2) sieving the blended material to form a sieved material;
(b3) blending the sieved material to form a blended/sieved material;
(b4) compacting the blended/sieved material to form a compacted material;
(b5) milling the compacted material to form a milled material; and
(b6) blending the milled material to form an amlodipine granulation.

19. The method of claim 16 further comprising the step of:
(d) film coating the bilayer solid dosage form.

20. A method of treating hypertension, congestive heart failure, angina, myocardial infarction, arteriosclerosis, diabetic nephropathy, diabetic cardiac myopathy, renal insufficiency, peripheral vascular disease, stroke, left ventricular hypertrophy, cognitive dysfunction, headache, or chronic heart failure, wherein the method comprises administering a solid dosage form of valsartan as defined in claim 1 to a subject in need of such treatment.

21. The method of treating according to claim 20, wherein the solid dosage form is orally administered to the subject.
